(19) United States
(12) Patent Application Publication
(10) Pub. No.: US 2023/0167075 A1
(43) Pub. Date: Jun. 1, 2023

(54) INTERLEUKIN INHIBITORS

(71) Applicant: UNIVERSITY OF GREENWICH, London (GB)

(72) Inventors: Mike LEACH, London (GB); Paul WILLIAMS, London (GB)

(21) Appl. No.: 17/921,036

(22) PCT Filed: Apr. 26, 2021
(86) PCT No.: PCT/GB2021/051004

§ 371 Date: Oct. 24, 2022

(30) Foreign Application Priority Data
Apr. 24, 2020 (GB) 2006076.0

(51) Int. Cl. C07D 253/075; C07D 239/49; A61P 37/02

(52) U.S. Cl. CPC C07D 253/075; C07D 239/49; A61P 37/02

(57) ABSTRACT

The present invention relates to diazine and triazine compounds having activity as Interleukin inhibitors, particularly Interleukin-1 beta, 2, 4, 6, 8, 13 and 17, and to the compounds for use in the treatment of associated disorders, particularly Alzheimer's Disease, Parkinson's Disease, Asthma, and solid organ transplant rejection.

### TECHNICAL FIELD

The present invention relates to diazine and triazine compounds having activity as Interleukin inhibitors, particularly Interleukin-1 beta, 2, 4, 6, 8, 13 and 17, and to the compounds for use in the treatment of associated disorders, particularly Alzheimer's Disease, Parkinson's Disease, Asthma, and solid organ transplantation rejection.

### BACKGROUND

WO2009090431A discloses triazines of the formula below, in which the A ring may be a sulphur containing heterocycle such as thienyl and benzothienyl, optionally substituted.

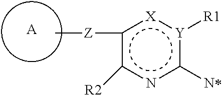

WO2009090431A further discloses triazines of the formula:

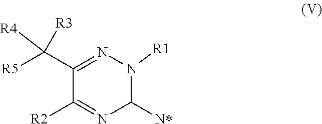

The compounds of WO2009090431A are stated to have activity as voltage dependent sodium channel blockers.

Foreman et al., Pharmacology, Biochemistry and Behaviour 89 (2008) 523-534, discuss a study of the compound JZP-4, which is stated to be a calcium and sodium channel blocker, in animal models for anticonvulsant, antimania and antidepressant activity. JZP-4 has the structure:

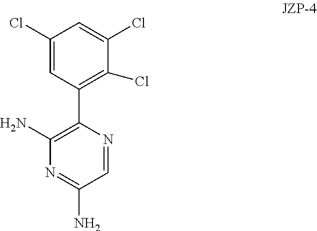 [SMILES: Nc1cnc(-c2cc(Cl)cc(Cl)c2Cl)c(N)n1]

GB735702B discusses 2,4-diaminopyrimidines and methods of preparing the same. The compounds are stated to be active in the treatment of malarial infection.

Inhibition of Interleukins is known to be useful in a number of diseases and conditions.

Inhibition of Interleukin-1 beta is useful in the treatment of inter alia epilepsy, particularly drug refractory epilepsy (Vezzani et al., 2019, Nature Reviews Neurology, 15(8), pp. 459-472 and Kumar et al., 2019, JCI Insight, 4(8)), Systemic juvenile arthritis (https://juvenilearthritisnews.com/arcalyst-rilonacept/) oncology, particularly breast (more particularly metastatic breast cancer), colon, lung, head and neck cancers, and melanomas (Tulotta and Otterwell, Endocrine-Related Cancer, 2018, 25(7), pp. R421-R434; and Baker et al., Frontiers in Immunology, 2019, 10). IL-1 beta is also useful in the treatment of Glaucoma, Stroke, Brain injury, diabetic retinopathy, Alzheimer's disease, and Multiple Sclerosis (Mendiola, A. and Cardona, A., 2017, Journal of Neural Transmission, 125(5), pp. 781-795); Acute brain injury (Brough et al., 2011. Trends in Pharmacological Sciences, 32(10), 617-622); Spinal cord Injury (Boato et al., 2013. Journal of Neuroinflammation, 10(1)); Motor neurone disease (Meissner et al., 2010. Proceedings of the National Academy of Sciences, 107(29), pp. 13046-13050); Parkinson's disease (Erekat and Al-Jarrah, 2018, Medical Science Monitor, 24, pp. 7524-7531); Neuropathic pain (Hung et al., 2017. Scandinavian Journal of Pain, 17(1), pp. 287-293); migraine (He et al., 2019. Journal of Neuroinflammation, 16(1); anxiety (McKim et al., 2017, Molecular Psychiatry, 23(6), pp. 1421-1431); Trigeminal autonomic cephalalgias (Neeb et al., 2016, The Journal of Headache and Pain, 17(1)); and inflammatory pain (Dinarello et al., 2012, Nature Reviews Drug Discovery, 11(8), 633-652).

Interleukin-2 inhibitors are known to have activity as immunosuppressive agents and anti-inflammatory agents and are therefore useful in reducing rejection of organ transplantation (Karahan et al, 2019, Transplantation Proceedings, 51(4), pp. 1074-1077). An IL-2 inhibitor has been approved by the FDA for the treatment of relapsing forms of multiple sclerosis (Pharmacy Today August 2016 Volume 22, Issue 8, Page 38).

Interleukin-6 inhibitors are known to be useful in the treatment of uveitis (Karkhur et al., J Ophthal Inflamm Infect 9, 17, 2019), Rheumatoid arthritis (Navarro et al., Seminars in arthritis and rheumatism, 2014, 43(4): p. 458-469), and systemic juvenile idiopathic arthritis (Yokota et al., Arthritis and rheumatism, 2005. 52(3): p. 818-825).

Interleukin-4 and 13 are key drivers of type 2 inflammation and IL-4 and IL-13 inhibitors are therefore useful potential treatments for diseases and conditions driven by allergic and other type 2 inflammation, including atopic dermatitis, asthma, chronic rhinosinusitis with nasal polyposis, eosinophilic esophagitis, chronic obstructive pulmonary disease, Idiopathic Pulmonary Fibrosis, Alopecia Areata, Pulmonary Tuberculosis, Hodgkin's disease, and food and environmental allergies. In particular, IL-4 is useful in the treatment of chronic asthma (Steinke and Borish, Respir Res. 2001; 2(2): 66-70); Atopic Dermatitis, chronic rhinosinusitis, Sleep Apnea and eczema (Junttila, Frontiers in Immunology, 2018, Vol 9, p 888). IL-13 is also useful in the treatment of Hodgkins Disease (Junttila, Frontiers in Immunology, 2018, Vol 9, p 888).

Interleukin-17 inhibitors are useful in the treatment of multiple sclerosis (Kolbinger et al., 2016, Current Drug Targets (2016) 17: 1882); ischemic stroke (Gelderblom et al., 2012, Blood, 120(18), 3793-3802); and neuropathic pain (Hung et al., A., 2017, Scandinavian Journal of Pain, 17(1), pp. 287-293).

### SUMMARY OF THE INVENTION

The invention provides a compound of the formula (I), or a salt, tautomer or solvate thereof;

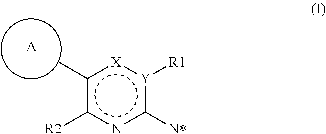

in which:

X is N and Y is C; or

X is C and Y is N; or

X and Y are both N:

A is a substituted 3 to 10 membered heterocycle comprising one, two or three sulphur atoms; said

heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups; or

A is a group

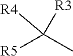

(wherein • indicates the point of attachment)

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

R3 is phenyl, xanthyl or naphthyl, each optionally substituted with 1 to 5 substituents selected from halogen or C₁-C₆alkoxy groups;

R4 is selected from hydrogen, C₁-C₆alkyl, C₃-C₈cycloalkyl, phenyl, xanthyl or naphthyl, wherein the phenyl or naphthyl may be optionally substituted with 1 to 5, preferably 2 to 5 substituents selected from halogen or C₁-C₆alkoxy groups;

R5 is hydrogen;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

The Invention further provides a method of treating a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis, comprising the step of administering to a subject in need thereof a therapeutically effective amount of a compound of formula (I) or a salt, tautomer or solvate thereof.

The invention further provides a compound of the formula (IA), or a salt, tautomer or solvate thereof;

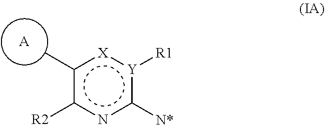

in which:

X is N and Y is C; or

X is C and Y is N

A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of a disorder or condition selected from epilepsy; multiple sclerosis; glaucoma and uveitis; cerebral traumas and cerebral ischaemias; stroke, head injury; spinal cord injury; surgical trauma; neurodegenerative disorders; motor neurone disease; Alzheimer's disease; Parkinson's disease; chronic inflammatory pain; neuropathic pain; migraine; bipolar disorder; mood, anxiety and cognitive disorders; schizophrenia; and trigeminal autonomic cephalalgias.

The Invention further provides a method of treating a disorder or condition selected from epilepsy; multiple sclerosis; glaucoma and uveitis; cerebral traumas and cerebral ischaemias; stroke, head injury; spinal cord injury; surgical trauma; neurodegenerative disorders; motor neurone disease; Alzheimer's disease; Parkinson's disease; chronic inflammatory pain; neuropathic pain; migraine; bipolar disorder; mood, anxiety and cognitive disorders; schizophrenia; and trigeminal autonomic cephalalgias, comprising the step of administering to a subject in need thereof a therapeutically effective amount of a compound of formula (IA) or a salt, tautomer or solvate thereof.

The invention further provides a compound of the formula (IA), or a salt, tautomer or solvate thereof;

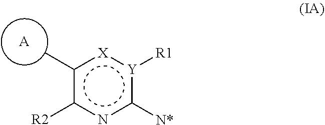

in which:

X is N and Y is C; or

X is C and Y is N

A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; provided that when X is C and Y is N, and A is thienyl optionally substituted with halogen, then R2 is not C₁-C₃ alkyl.

The Invention further provides a compound of the formula (IB), or a salt, tautomer or solvate thereof,

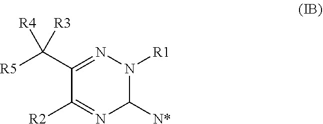

in which

R3 is phenyl, xanthyl or naphthyl, each optionally substituted with 1 to 5 substituents selected from halogen or C₁-C₆alkoxy groups;

R4 is selected from hydrogen, C₁-C₆alkyl, C₃-C₈cycloalkyl, phenyl, xanthyl or naphthyl, wherein the phenyl or naphthyl may be optionally substituted with 1 to 5, preferably 2 to 5 substituents selected from halogen or C₁-C₆alkoxy groups;

R5 is hydrogen;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

N* is amino when R1 is hydrogen or ═NH when R1 is a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

The Invention further provides a method of treating a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis, comprising the step of administering to a subject in need thereof a therapeutically effective amount of a compound of formula (IB) or a salt, tautomer or solvate thereof.

### BRIEF DESCRIPTION OF THE DRAWINGS

FIG. 1 illustrates the inhibitory effect of 3,5-diamino-6-[2-(3,4,5-trichlorothienyl)]-1,2,4-triazine on Interleukin (IL)-1 beta.

FIG. 2 illustrates the inhibitory effect of 3,5-diamino-6-[2-(3,4,5-trichlorothienyl)]-1,2,4-triazine on interleukin (IL)-6.

FIG. 3 illustrates the inhibitory effect of 3,5-diamino-6-[2-(3,4,5-trichlorothienyl)]-1,2,4-triazine on interleukin (IL)-8.

FIG. 4 illustrates the inhibitory effect of 3,5-diamino-6-[2-(3,4,5-trichlorothienyl)]-1,2,4-triazine on interleukin (IL)-17A.

FIG. 5 illustrates the inhibitory effect of 3,5-Diamino-6-(diphenylmethyl)-1,2,4-triazine on interleukin (IL)-17A.

FIG. 6 illustrates the inhibitory effect of 3,5-Diamino-6-(diphenylmethyl)-1,2,4-on interleukin (IL)-8.

FIG. 7 illustrates the inhibitory effect of 3,5-Diamino-6-(diphenylmethyl)-1,2,4-on interleukin (IL)-6.

### DETAILED DESCRIPTION OF THE INVENTION

It will be recognised that formulae (IA) and (IB) are sub-formulae of formula (I).

The compounds of the formulae (I), (IA) and (IB) are inhibitors of interleukin 1 beta, 2, 4, 6, 8, 13 or 17 and therefore are useful in the treatment of a number of disorders and conditions.

As Embodiment 1, the invention provides a compound of the formula (I), or a salt, tautomer or solvate thereof;

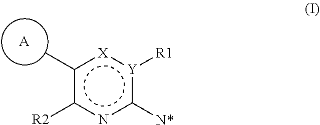

in which:

X is N and Y is C; or

X is C and Y is N; or

X and Y are both N:

A is a substituted 3 to 10 membered heterocycle comprising one, two or three sulphur atoms; said

heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups; or

A is a group

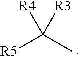

(wherein • indicates the point of attachment)

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

R3 is phenyl, xanthyl or naphthyl, each optionally substituted with 1 to 5, preferably 2 to 5 substituents selected from halogen or C₁-C₆alkoxy groups;

R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups;

R5 is hydrogen;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

The Compound for use as defined in Embodiment 1 is preferably a compound selected from:
3,5-Diamino-6-(2-thienyl)-1,2,4-triazine;
3,5-Diamino-6-(3-thienyl)-1,2,4-triazine;
3,5-Diamino-6-[3-(2,5 dichlorothienyl)]-1,2,4-triazine;
3,5-Diamino-6-[2-(3,4,5 trichlorothienyl)]-1,2,4-triazine;
5(3)-Amino-6-(2-thienyl)-2,3(2,5)-dihydro-3(5)-imino-2-methyl-1,2,4-triazine;
5(3)-Amino-6-(2-thienyl)-2,3(2,5)-dihydro-3(5)-imino-2-ethyl-1,2,4-triazine;
5(3)-Amino-6-[3-(2,5-dichlorothienyl)]-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-ethyl-1,2,4-triazine;
3,5-Diamino-6-[2-(4,5-dibromothienyl)]-1,2,4-triazine;
3,5-Diamino-6-[2-(5-bromothienyl)]-1,2,4-triazine;
3,5-Diamino-6-[2-(3-bromothienyl)]-1,2,4-triazine;
3,5-Diamino-6-[2-(5-chlorothienyl)]-1,2,4-triazine;
3,5-Diamino-6-[2-(benzo[b]thienyl)]-1,2,4-triazine;
3,5-Diamino-6-[2-(3-chlorobenzo[b]thienyl)]-1,2,4-triazine.
2,6-Diamino-3-(2-thienyl)-pyrazine (also referred to as 3,5-Diamino-6-(2-thienyl)-pyrazine);
2,4-Diamino-5-(2-thienyl)-pyrimidine (also referred to as 3,5-Diamino-6-(2-thienyl)-pyrimidine);
2,6-Diamino-3-(3-thienyl)-pyrazine (also referred to as 3,5-Diamino-6-(3-thienyl)-pyrazine);
2,4-Diamino-5-(3-thienyl)-pyrimidine (also referred to as 3,5-Diamino-6-(3-thienyl)-pyrimidine);
2,6-Diamino-3-[3-(2,5 dichlorothienyl)]-pyrazine (also referred to as 3,5-Diamino-6-[3-(2,5 dichlorothienyl)]-pyrazine);
2,4-Diamino-5-[3-(2,5 dichlorothienyl)]-pyrimidine (also referred to as 3,5-Diamino-6-[3-(2,5 dichlorothienyl)]-pyrimidine);
2,6-Diamino-3-[2-(3,4,5 trichlorothienyl)]-pyrazine (also referred to as 3,5-Diamino-6-[2-(3,4,5 trichlorothienyl)]-pyrazine);
2,4-Diamino-5-[2-(3,4,5 trichlorothienyl)]-pyrimidine (also referred to as 3,5-Diamino-6-[2-(3,4,5 trichlorothienyl)]-pyrimidine);
2(6)-Amino-3-(2-thienyl)-2,3(2,5)-dihydro-6(2)-imino-5-methyl-pyrazine (also referred to as 5(3)-Amino-6-(2-thienyl)-2,3(2,5)-dihydro-3(5)-imino-2-methyl-pyrazine);
4(2)-Amino-5-(2-thienyl)-2,3(2,5)-dihydro-2(4)-imino-1-methyl-pyrimidine (also referred to as 5(3)-Amino-6-(2-thienyl)-2,3(2,5)-dihydro-3(5)-imino-2-methyl-pyrimidine);
2(6)-Amino-3-(2-thienyl)-2,3(2,5)-dihydro-6(2)-imino-5-ethyl-pyrazine (also referred to as 5(3)-Amino-6-(2-thienyl)-2,3(2,5)-dihydro-3(5)-imino-2-ethyl-pyrazine);
4(2)-Amino-5-(2-thienyl)-2,3(2,5)-dihydro-2(4)-imino-1-ethyl-pyrimidine (also referred to as 5(3)-Amino-6-(2-thienyl)-2,3(2,5)-dihydro-3(5)-imino-2-ethyl-pyrimidine);
2(6)-Amino-3-[3-(2,5-dichlorothienyl)]-2,3(2,5)-dihydro-6(2)-imino-5-methyl-pyrazine (also referred to as 5(3)-Amino-6-[3-(2,5-dichlorothienyl)]-2,3(2,5)-dihydro-3(5)-imino-2-methyl-pyrazine);
4(2)-Amino-5-[3-(2,5-dichlorothienyl)]-2,3(2,5)-dihydro-2(4)-imino-1-methyl-pyrimidine (also referred to as 5(3)-Amino-6-[3-(2,5-dichlorothienyl)]-2,3(2,5)-dihydro-3(5)-imino-2-methyl-pyrimidine);
2(6)-Amino-3-{2-(3,4,5-trichloro)thienyl}-2,3(2,5)-dihydro-6(2)-imino-5-methyl-pyrazine (also referred to as 5(3)-Amino-6-{2-(3,4,5-trichloro)thienyl}-2,3(2,5)-dihydro-3(5)-imino-2-methyl-pyrazine);
4(2)-Amino-5-{2-(3,4,5-trichloro)thienyl}-2,3(2,5)-dihydro-2(4)-imino-1-methyl-pyrimidine (also referred to as 5(3)-Amino-6-{2-(3,4,5-trichloro)thienyl}-2,3(2,5)-dihydro-3(5)-imino-2-methyl-pyrimidine);
2(6)-Amino-3-{2-(3,4,5-trichloro)thienyl}-2,3(2,5)-dihydro-6(2)-imino-5-ethyl-pyrazine (also referred to as 5(3)-Amino-6-{2-(3,4,5-trichloro)thienyl}-2,3(2,5)-dihydro-3(5)-imino-2-ethyl-pyrazine);
4(2)-Amino-5-{2-(3,4,5-trichloro)thienyl}-2,3(2,5)-dihydro-2(4)-imino-1-ethyl-pyrimidine (also referred to as 5(3)-Amino-6-{2-(3,4,5-trichloro)thienyl}-2,3(2,5)-dihydro-3(5)-imino-2-ethyl-pyrimidine);
2,6-Diamino-3-[2-(4,5-dibromothienyl)]-pyrazine (also referred to as 3,5-Diamino-6-[2-(4,5-dibromothienyl)]-pyrazine);
2,4-Diamino-5-[2-(4,5-dibromothienyl)]-pyrimidine (also referred to as 3,5-Diamino-6-[2-(4,5-dibromothienyl)]-pyrimidine);
2,6-Diamino-3-[2-(5-bromothienyl)]-pyrazine (also referred to as 3,5-Diamino-6-[2-(5-bromothienyl)]-pyrazine);
2,4-Diamino-5-[2-(5-bromothienyl)]-pyrimidine (also referred to as 3,5-Diamino-6-[2-(5-bromothienyl)]-pyrimidine);
2,6-Diamino-3-[2-(3-bromothienyl)]-pyrazine (also referred to as 3,5-Diamino-6-[2-(3-bromothienyl)]-pyrazine);
2,4-Diamino-5-[2-(3-bromothienyl)]-pyrimidine (also referred to as 3,5-Diamino-6-[2-(3-bromothienyl)]-pyrimidine);
2,6-Diamino-3-[2-(5-chlorothienyl)]-pyrazine (also referred to as 3,5-Diamino-6-[2-(5-chlorothienyl)]-pyrazine);
2,4-Diamino-5-[2-(5-chlorothienyl)]-pyrimidine (also referred to as 3,5-Diamino-6-[2-(5-chlorothienyl)]-pyrimidine);
2,6-Diamino-3-[2-(benzo[b]thienyl)]-pyrazine (also referred to as 3,5-Diamino-6-[2-(benzo[b]thienyl)]-pyrazine);
2,4-Diamino-5-[2-(benzo[b]thienyl)]-pyrimidine (also referred to as 3,5-Diamino-6-[2-(benzo[b]thienyl)]-pyrimidine);
2,6-Diamino-3-[2-(3-chlorobenzo[b]thienyl)]-pyrazine (also referred to as 3,5-Diamino-6-[2-(3-chlorobenzo[b]thienyl)]-pyrazine);
2,4-Diamino-5-[2-(3-chlorobenzo[b]thienyl)]-pyrimidine (also referred to as 3,5-Diamino-6-[2-(3-chlorobenzo[b]thienyl)]-pyrimidine);
3,5-Diamino-6-(diphenylmethyl)-1,2,4-triazine;
2,6-diamino-3-(diphenylmethyl)-pyrazine;
2,4-diamino-5-(diphenylmethyl)-pyrimidine;
3,5-Diamino-6-(1-cyclopentyl-1-phenyl-methyl)-1,2,4-triazine;
2,6-Diamino-3-(1-cyclopentyl-1-phenyl-methyl)-pyrazine;
2,4-Diamino-5-(1-cyclopentyl-1-phenyl-methyl)-pyrimidine;
3,5-Diamino-6-[1-(6-methoxynaphthalene)methyl]-1,2,4-triazine;
2,6-Diamino-3-[1-(6-methoxynaphthalene)methyl]-pyrazine;
2,4-Diamino-5-[1-(6-methoxynaphthalene)methyl]-pyrimidine;
3,5-Diamino-6-[1-(6-methoxynaphthalene)ethyl]-1,2,4-triazine;
2,6-Diamino-3-[1-(6-methoxynaphthalene)ethyl]-pyrazine;
2,4-Diamino-5-[1-(6-methoxynaphthalene)ethyl]-pyrimidine;
3,5-Diamino-6-(1-isopropyl-1-phenylmethyl)-1,2,4-triazine;
2,6-Diamino-3-(1-isopropyl-1-phenylmethyl)-pyrazine;
2,4-Diamino-5-(1-isopropyl-1-phenylmethyl)-pyrimidine;
3,5-Diamino-6-(9-xanthyl)-1,2,4-triazine;
2,6-Diamino-3-(9-xanthyl)-pyrazine;
2,4-Diamino-5-(9-xanthyl)-pyrimidine;
3,5-Diamino-6-[1,1 bis-(4-chlorophenyl)methyl]-1,2,4-triazine;
2,6-Diamino-3-[1,1 bis-(4-chlorophenyl)methyl]-pyrazine;
2,4-Diamino-5-[1,1 bis-(4-chlorophenyl)methyl]-pyrimidine;
3,5-Diamino-6-[1,1-bis-(4-fluorophenyl)methyl]-1,2,4-triazine;
2,6-Diamino-3-[1,1-bis-(4-fluorophenyl)methyl]-pyrazine;
2,4-Diamino-5-[1,1-bis-(4-fluorophenyl)methyl]-pyrimidine;
3,5-Diamino-6-{1-(4-chlorophenoxy)-1-methyl}ethyl-1,2,4-triazine;
2,6-Diamino-3-{1-(4-chlorophenoxy)-1-methyl}ethyl-pyrazine; and
2,4-Diamino-5-{1-(4-chlorophenoxy)-1-methyl}ethyl-pyrimidine;

or a salt, tautomer or solvate thereof.

Certain pyrazine and pyrimidine compounds listed above, alongside the IUPAC nomenclature, are numbered in conformity with the numbering used for the triazine embodiments, i.e., the X atom is at the 1 position, the Y atom is at the 2 position, the C atom substituted by N* is at the 3 position and so on, with the C atom substituted by the A ring at the 6-position.

As Embodiment 2, the invention provides a compound of Formula (IA), or a salt, tautomer or solvate thereof

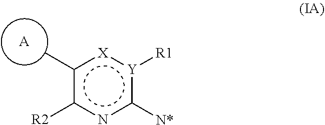

in which:

X is N and Y is C; or

X is C and Y is N

A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of epilepsy; multiple sclerosis; glaucoma and uveitis; cerebral traumas and cerebral ischaemias; stroke, head injury; spinal cord injury; surgical trauma; neurodegenerative disorders; motor neurone disease; Alzheimer's disease; Parkinson's disease; chronic inflammatory pain; neuropathic pain; migraine; bipolar disorder; mood, anxiety and cognitive disorders; schizophrenia; and trigeminal autonomic cephalalgias.

As Embodiment 3, the invention provides a compound of Formula (IA), or a salt, tautomer or solvate thereof, for use as defined in Embodiment 2, wherein A is thienyl, or benzothienyl.

As Embodiment 4, the invention provides a compound of Formula (IA) or a salt, tautomer or solvate thereof, for use as defined in any previous Embodiment, wherein A is substituted with one or more substituents selected from halogen, C₁₋₆alkyl, C₁₋₆alkoxy, haloC₁₋₆alkyl and haloC₁₋₆ alkoxy.

As Embodiment 5, the invention provides a compound of Formula (IA) or a salt, tautomer or solvate thereof, for use as defined in any previous Embodiment, wherein A is substituted with 1, 2, or 3 chlorine or bromine atoms.

As Embodiment 6, the invention provides a compound of Formula (IA), or a salt, tautomer or solvate thereof,

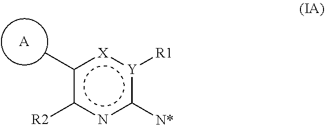

in which:

X is N and Y is C; or

X is C and Y is N

A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

and

N* is ═NH when R1 is hydrogen or a substituent group; or

N* is a group NRaRb where Ra and Rb are independently H or an alkyl group; or

N* is a piperazinyl ring, optionally substituted with one or more halogen or C₁-C₆alkoxy groups; provided that when X is C and Y is N, and A is thienyl optionally substituted with halogen, then R2 is not C₁-C₃ alkyl.

As Embodiment 7, the invention provides a compound of Formula (I) which is selected from:
2,6-Diamino-3-(2-thienyl)-pyrazine (also referred to as 3,5-Diamino-6-(2-thienyl)-pyrazine);
2,4-Diamino-5-(2-thienyl)-pyrimidine (also referred to as 3,5-Diamino-6-(2-thienyl)-pyrimidine);
2,6-Diamino-3-(3-thienyl)-pyrazine (also referred to as 3,5-Diamino-6-(3-thienyl)-pyrazine);
2,4-Diamino-5-(3-thienyl)-pyrimidine (also referred to as 3,5-Diamino-6-(3-thienyl)-pyrimidine);
2,6-Diamino-3-[3-(2,5 dichlorothienyl)]-pyrazine (also referred to as 3,5-Diamino-6-[3-(2,5 dichlorothienyl)]-pyrazine);
2,4-Diamino-5-[3-(2,5 dichlorothienyl)]-pyrimidine (also referred to as 3,5-Diamino-6-[3-(2,5 dichlorothienyl)]-pyrimidine);
2,6-Diamino-3-[2-(3,4,5 trichlorothienyl)]-pyrazine (also referred to as 3,5-Diamino-6-[2-(3,4,5 trichlorothienyl)]-pyrazine);
2,4-Diamino-5-[2-(3,4,5 trichlorothienyl)]-pyrimidine (also referred to as 3,5-Diamino-6-[2-(3,4,5 trichlorothienyl)]-pyrimidine);
2(6)-Amino-3-(2-thienyl)-2,3(2,5)-dihydro-6(2)-imino-5-methyl-pyrazine (also referred to as 5(3)-Amino-6-(2-thienyl)-2,3(2,5)-dihydro-3(5)-imino-2-methyl-pyrazine);
4(2)-Amino-5-(2-thienyl)-2,3(2,5)-dihydro-2(4)-imino-1-methyl-pyrimidine (also referred to as 5(3)-Amino-6-(2-thienyl)-2,3(2,5)-dihydro-3(5)-imino-2-methyl-pyrimidine);
2(6)-Amino-3-(2-thienyl)-2,3(2,5)-dihydro-6(2)-imino-5-ethyl-pyrazine (also referred to as 5(3)-Amino-6-(2-thienyl)-2,3(2,5)-dihydro-3(5)-imino-2-ethyl-pyrazine);
4(2)-Amino-5-(2-thienyl)-2,3(2,5)-dihydro-2(4)-imino-1-ethyl-pyrimidine (also referred to as 5(3)-Amino-6-(2-thienyl)-2,3(2,5)-dihydro-3(5)-imino-2-ethyl-pyrimidine);
2(6)-Amino-3-[3-(2,5-dichlorothienyl)]-2,3(2,5)-dihydro-6(2)-imino-5-methyl-pyrazine (also referred to as 5(3)-Amino-6-[3-(2,5-dichlorothienyl)]-2,3(2,5)-dihydro-3(5)-imino-2-methyl-pyrazine);
4(2)-Amino-5-[3-(2,5-dichlorothienyl)]-2,3(2,5)-dihydro-2(4)-imino-2-methyl-pyrimidine (also referred to as 5(3)-Amino-6-[3-(2,5-dichlorothienyl)]-2,3(2,5)-dihydro-3(5)-imino-2-methyl-pyrimidine);
2(6)-Amino-3-{2-(3,4,5-trichloro)thienyl}-2,3(2,5)-dihydro-6(2)-imino-5-methyl-pyrazine (also referred to as 5(3)-Amino-6-{2-(3,4,5-trichloro)thienyl}-2,3(2,5)-dihydro-3(5)-imino-2-methyl-pyrazine);
4(2)-Amino-5-{2-(3,4,5-trichloro)thienyl}-2,3(2,5)-dihydro-2(4)-imino-1-methyl-pyrimidine (also referred to as 5(3)-Amino-6-{2-(3,4,5-trichloro)thienyl}-2,3(2,5)-dihydro-3(5)-imino-2-methyl-pyrimidine);
2(6)-Amino-3-{2-(3,4,5-trichloro)thienyl}-2,3(2,5)-dihydro-6(2)-imino-5-ethyl-pyrazine (also referred to as 5(3)-Amino-6-{2-(3,4,5-trichloro)thienyl}-2,3(2,5)-dihydro-3(5)-imino-2-ethyl-pyrazine);
4(2)-Amino-5-{2-(3,4,5-trichloro)thienyl}-2,3(2,5)-dihydro-2(4)-imino-2-ethyl-pyrimidine (also referred to as 5(3)-Amino-6-{2-(3,4,5-trichloro)thienyl}-2,3(2,5)-dihydro-3(5)-imino-2-ethyl-pyrimidine);
2,6-Diamino-3-[2-(4,5-dibromothienyl)]-pyrazine (also referred to as 3,5-Diamino-6-[2-(4,5-dibromothienyl)]-pyrazine);
2,4-Diamino-5-[2-(4,5-dibromothienyl)]-pyrimidine (also referred to as 3,5-Diamino-6-[2-(4,5-dibromothienyl)]-pyrimidine);
2,6-Diamino-3-[2-(5-bromothienyl)]-pyrazine (also referred to as 3,5-Diamino-6-[2-(5-bromothienyl)]-pyrazine);
2,4-Diamino-5-[2-(5-bromothienyl)]-pyrimidine (also referred to as 3,5-Diamino-6-[2-(5-bromothienyl)]-pyrimidine);
2,6-Diamino-3-[2-(3-bromothienyl)]-pyrazine (also referred to as 3,5-Diamino-6-[2-(3-bromothienyl)]-pyrazine);
2,4-Diamino-5-[2-(3-bromothienyl)]-pyrimidine (also referred to as 3,5-Diamino-6-[2-(3-bromothienyl)]-pyrimidine);
2,6-Diamino-3-[2-(5-chlorothienyl)]-pyrazine (also referred to as 3,5-Diamino-6-[2-(5-chlorothienyl)]-pyrazine);
2,4-Diamino-5-[2-(5-chlorothienyl)]-pyrimidine (also referred to as 3,5-Diamino-6-[2-(5-chlorothienyl)]-pyrimidine);
2,6-Diamino-3-[2-(benzo[b]thienyl)]-pyrazine (also referred to as 3,5-Diamino-6-[2-(benzo[b]thienyl)]-pyrazine);
2,4-Diamino-5-[2-(benzo[b]thienyl)]-pyrimidine (also referred to as 3,5-Diamino-6-[2-(benzo[b]thienyl)]-pyrimidine);
2,6-Diamino-3-[2-(3-chlorobenzo[b]thienyl)]-pyrazine (also referred to as 3,5-Diamino-6-[2-(3-chlorobenzo[b]thienyl)]-pyrazine); and
2,4-Diamino-5-[2-(3-chlorobenzo[b]thienyl)]-pyrimidine (also referred to as 3,5-Diamino-6-[2-(3-chlorobenzo[b]thienyl)]-pyrimidine);

or a salt, tautomer or solvate thereof.

Certain pyrazine and pyrimidine compounds listed above, alongside the IUPAC nomenclature, are numbered in conformity with the numbering used for the triazine embodiments, i.e., the X atom is at the 1 position, the Y atom is at the 2 position, the C atom substituted by N* is at the 3 position and so on, with the C atom substituted by the A ring at the 6-position.

The compounds of Formula (I) have been found to inhibit interleukin-1 beta and other Interleukins are therefore useful in the treatment of a number of disorders and conditions.

As Embodiment 8, the invention provides a pharmaceutical composition comprising a compound of Formula (IA), or a salt, tautomer or solvate thereof, as defined in Embodiment 6 or 7, and a pharmaceutically acceptably excipient.

As Embodiment 9, the invention provides a compound of Formula (I), or a salt, tautomer or solvate thereof, as defined in Embodiment 6 or 7, for use as a medicament.

As Embodiment 10, the invention provides a method of treating a subject disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis; comprising the step of administering to a subject in need thereof a therapeutically effective amount of a compound of formula (I) or a salt, tautomer or solvate thereof, as defined in any one of Embodiments 1 to 7.

The invention further provides a compound of Formula (IA), or a salt, tautomer thereof, as defined in Embodiments 6 and 7, for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis; or epilepsy; multiple sclerosis; glaucoma and uveitis; cerebral traumas and cerebral ischaemias; stroke, head injury; spinal cord injury; surgical trauma; neurodegenerative disorders; motor neurone disease; Alzheimer's disease; Parkinson's disease; chronic inflammatory pain; neuropathic pain; migraine; bipolar disorder; mood, anxiety and cognitive disorders; schizophrenia; and trigeminal autonomic cephalalgias.

Compounds of Formula (IB) have been found to have activity as inhibitors of Interleukin-2, 4 and 13, and are therefore useful in the treatment of a number of disease or conditions.

As Embodiment 11, the invention provides a compound of Formula (I), or a salt, tautomer or solvate thereof, wherein

A is a group

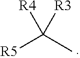

(wherein • indicates the point of attachment)

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

R3 is phenyl, xanthyl or naphthyl, each optionally substituted with 2 to 5 substituents selected from halogen or C₁-C₆alkoxy groups;

R4 is selected from hydrogen, C₁-C₆alkyl, C₃-C₈cycloalkyl, phenyl, xanthyl or naphthyl, wherein the phenyl or naphthyl may be optionally substituted with 1 to 5, preferably 2 to 5 substituents selected from halogen or C₁-C₆alkoxy groups;

R5 is hydrogen;

for use as defined in Embodiment 1.

As Embodiment 12, the invention provides a compound of Formula (IB) or a salt, tautomer or solvate thereof

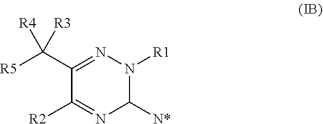

in which

R3 is phenyl, xanthyl or naphthyl, each optionally substituted with 1 to 5 substituents selected from halogen or C₁-C₆alkoxy groups;

R4 is selected from hydrogen, C₁-C₆alkyl, C₃-C₈cycloalkyl, phenyl, xanthyl or naphthyl, wherein the phenyl or naphthyl may be optionally substituted with 1 to 5, preferably 2 to 5 substituents selected from halogen or C₁-C₆alkoxy groups;

R5 is hydrogen;

R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;

R2 is amino, C₁₋₁₀ alkyl or phenyl;

N* is amino when R1 is hydrogen or ═NH when R1 is a substituent group; or

N* is a group NRaRb where Ra and Rb are independently H or an alkyl group; or

N* is a piperazinyl ring, optionally substituted with one or more halogen or C₁-C₆alkoxy groups; for use in the treatment of a disease or conditions selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

As Embodiment 12, the invention provides a compound of Formula (IB), or a salt, tautomer or solvate thereof, for use as defined in Embodiment 10, wherein R3 is phenyl, optionally substituted with 2 or 3 substituents selected from one or more halogen or C₁-C₆alkoxy groups; and R4 is selected from C₁-C₆alkyl, C₃-C₈cycloalkyl, phenyl, wherein the phenyl may be optionally substituted with 1 to 3 substituents selected from one or more halogen or C₁-C₆alkoxy groups.

As Embodiment 13, the invention provides a compound of Formula (IB), or a salt, tautomer or solvate thereof, for use as defined in Embodiment 11, wherein the compound is selected from:
3,5-Diamino-6-(diphenylmethyl)-1,2,4-triazine;
3,5-Diamino-6-(1-cyclopentyl-1-phenyl-methyl)-1,2,4-triazine;
3,5-Diamino-6-[1-(6-methoxynaphthalene)methyl]-1,2,4-triazine;
3,5-Diamino-6-[1-(6-methoxynaphthalene)ethyl]-1,2,4-triazine;
3,5-Diamino-6-(1-isopropyl-1-phenylmethyl)-1,2,4-triazine
3,5-Diamino-6-(9-xanthyl)-1,2,4-triazine;
3,5-Diamino-6-[1,1 bis-(4-chlorophenyl)methyl]-1,2,4-triazine;
3,5-Diamino-6-[1,1-bis-(4-fluorophenyl)methyl]-1,2,4-triazine;

and
3,5-Diamino-6-{1-(4-chlorophenoxy)-1-methyl}ethyl-1,2,4-triazine; or

a salt, tautomer or solvate thereof.

The use of salts of the compounds of formulae (I), (IA) and (IB) form an aspect of this invention. Preferred salts are pharmaceutically acceptable acid addition salts. Suitable pharmaceutically acceptable acid addition salts include those formed with both organic and inorganic acids, for example from hydrochloric, sulphuric, citric, tartaric, phosphoric, lactic, pyruvic, acetic, malonic, succinic, oxalic, fumaric, maleic, oxaloacetic, methanesulphonic, p-toluenesulphonic, benzene-sulphonic, glutamic, naphthoic, and isethionic acids. Ethanesulfonate, malate, mandalate, benzoate, and salicylate salts are also suitable. Base addition salts also form an aspect of the invention.

In preparation of the compounds of formula (I), (IA) or (IB), the compound or its salt may be obtained as a solvate of the reaction solvent or crystallisation solvent or a component thereof. Use of such solvates forms another aspect of this invention. Suitable pharmaceutically acceptable solvates include hydrates.

Certain compounds of structure (1), (IA) or (IB) have chiral centres and may occur as racemates, racemic mixtures and as individual enantiomers or diastereomers. All such isomeric forms are included within the present invention. Also included within the scope of the invention are all geometric isomers of the compound of formula (I), (IA) or (IB) whether as individual isomers or mixtures thereof. Thus, compounds of structure (1), (IA) or (IB) in the trans and cis configuration form a further aspect of the invention; also all other tautomeric form of structure (1), (IA) or (IB), including mixtures thereof. Furthermore, some of the crystalline forms of the compounds of structure (IA) or (IB) may exist as polymorphs, which are all included in the present invention. Compounds of Formula (IA) may be prepared by procedures analogous to those described in EP-0372934A. The reactants of formulae (II), (IV) and (V) disclosed in EP-0372934A may be replaced with corresponding sulphur containing heterocyclic analogues in order to prepare the presently claimed compounds.

Compounds of Formula (IB) may be prepared according to the procedures described in WO2009090431A.

The preparation of specific compounds mentioned above is illustrated later in this specification. Related compounds within the scope of the invention may be prepared by obvious or routine variations of the disclosed processes, using appropriate starting materials to introduce the desired substituents and moieties of compounds within the scope of formula (IA).

Salts of compounds of formula (IA) and (IB) may be obtained by the presence of a residual acid in the preparative process. Alternatively, salts may be prepared by mixing the compound of formula (IA) or (IB) as the free base with a pharmaceutically acceptable acid in a suitable solvent, and removing the solvent to recover the salt, or crystallising the salt from the solvent.

In a further aspect, the present invention provides pharmaceutical compositions for the treatment of disorders such those detailed in the Embodiments above comprising a compound of formula (I), or a pharmaceutically acceptable salt, tautomer or solvate thereof, in admixture with a pharmaceutically acceptable carrier.

The compounds of formula (I) will be present in the compositions of the present invention in an effective unit dosage form, that is to say in an amount sufficient to be effective against the disorders in vivo.

The pharmaceutically acceptable carriers present in the compositions of the present invention may be materials conventionally used for the purpose of administering the medicament. These may be liquid or solid materials, which are otherwise inert or medically acceptable and are compatible with the active ingredients.

These pharmaceutical compositions may be given orally or parenterally, for example as a suppository, ointment, cream, powder or trans-dermal patch. However, oral administration and intravenous injection of the compositions are preferred. For oral administration, fine powders or granules will contain diluting, dispersing and/or surface active agents, and may be presented in draught, in water or in a syrup, in capsules or sachets in the dry state or in non-aqueous suspension wherein suspending agents may be included, or in a suspension in water or syrup. Where desirable or necessary, flavouring, preserving, suspending, or thickening agents can be included. Dry powders or granules may be compressed to form a tablet or contained in a capsule. For injection, the compounds may be presented in sterile aqueous injection solutions which may contain anti-oxidants or buffers.

The free base or a salt or solvate thereof may also be administered in its pure form unassociated with other additives in which case a capsule or sachet is the preferred carrier. Alternatively, the active compound may be presented in a pure form as an effective unit dosage, for instance compressed as a tablet or the like. Other compounds which may be included are, for example, medically inert ingredients, e.g., solid and liquid diluents such as lactose, starch, or calcium phosphate for tablet or capsules; olive oil or ethyl oleate for soft capsules; and water or vegetable oil for suspensions or emulsions; lubricating agents such as talc or magnesium stearate; gelling agents such as colloidal clays; thickening agents such as gum tragacanth or sodium alginate; and other therapeutically acceptable accessory ingredients such as humectants, preservatives, buffers, and antioxidants which are useful as carriers in such formulations.

Tablets or other forms of presentation provided in discrete units may conveniently contain an amount of compound of formula I which is effective at such dosage or as a multiple of the same, for instance units containing 5 mg to 500 mg, usually around 10 mg to 250 mg.

The pharmaceutical compositions of the present invention may be prepared by the admixture of a compound of formula (I) with a pharmaceutically acceptable carrier. Conventional pharmaceutical excipients may be admixed as required. Example of suitable formulations are given in the above-mentioned U.S. Pat. No. 4,649,139.

The present invention provides a method of treatment of disorders in mammals that are susceptible to inhibition of interleukin, particularly interleukin 1-beta, 2, 4, 6, 8, 13 and 17 and particularly disorders such as epilepsy, multiple sclerosis, glaucoma and uevitis, cerebral traumas and cerebral ischaemias, stroke, head injury, spinal cord injury, surgical trauma, neurodegenerative disorders, motorneurone disease, Alzheimers disease, Parkinsons disease, chronic inflammatory pain, neuropathic pain, migraine, bipolar disorder, mood, anxiety and cognitive disorders, schizophrenia and trigeminal autonomic cephalalgias; for treatment of mammalian cancers; and for treatment of malaria; or disorders such as asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis; by the administration of a non-toxic effective amount of a compound of formula (I) or a pharmaceutically acceptable salt, tautomer or solvate thereof, or a composition as hereinbefore defined.

The present invention also provides of a compound of formula (I) or a pharmaceutically acceptable salt, tautomer or solvate thereof, or a composition as hereinbefore defined. for, or for the preparation of a medicament for, treatment of disorders in mammals that are susceptible to inhibition of interleukin, particularly interleukin 1-beta, 2, 4, 6, 8, 13 and 17 and particularly disorders such as epilepsy, multiple sclerosis, glaucoma and uevitis, cerebral traumas and cerebral ischaemias, stroke, head injury, spinal cord injury, surgical trauma, neurodegenerative disorders, motorneurone disease, Alzheimer's disease, Parkinson's disease, chronic inflammatory pain, neuropathic pain, migraine, bipolar disorder, mood, anxiety and cognitive disorders, schizophrenia and trigeminal autonomic cephalalgias; for treatment of mammalian cancers; and for treatment of malaria; or disorders such as asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

As indicated above, the compounds of formula (I) are generally useful in treating such disorders by oral administration or intravenous injection. The compounds of formula (I) are normally administered at a dose of from 0.01 mg/kg to 20 mg/kg per day, preferably 0.1 to 5.0 mg/kg per day.

### EXPERIMENTAL

The compounds of formulae (I), (IA) and (IB) may be prepared according to the methods disclosed in WO2009/090431A1, using the appropriate starting materials.

### Example 1: 2,4-Diamino-5-(diphenylmethyl)-pyrimidine

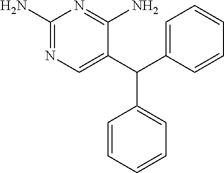 [SMILES: Nc1ncc(C(c2ccccc2)c2ccccc2)c(N)n1]

Formula: C₁₇H₁₆N₄

Molecular weight: 276.34

LCMS: m/z=277.20, consistent for protonated parent ion (M+H)⁺

¹H NMR (DMSO-d6): The ¹H NMR spectrum was found to be consistent with the above structure.

Purity: >99% by HPLC

The compounds of formulae (I), (IA) and (IB) may be investigated for inhibition of pro-inflammatory cytokines Interleukin (IL)-1p, 2, 4, 6, 8, 13 and 17A, in peripheral blood mononuclear cells (PBMCs) isolated from fresh human buffy coats by centrifugation on Lymphoprep™ (Stemcell Technologies). All human cells are grown in cell culture medium RPMI-1640 supplemented with 1% penicillin/streptomycin and 5% heat inactivated fetal bovine serum.

PBMCs stimulated with LPS (Salmonella enterica serotype typhimurium) are incubated for 24 hours containing the compounds under investigation, reconstituted in dimethyl sulfoxide (DMSO). The secreted levels of Interleukin-1(3 is measured in the cell culture supernatant using a cytometric bead array and the cell viability is quantified using Trypan blue.

PBMCs stimulated with PMA/ionomycin are incubated for 24 hours containing the compounds under investigation, reconstituted in DMSO. The secreted levels of Interleukin-17A are measured in the cell culture supernatant using a cytometric bead array and the cell viability is quantified using Trypan blue.

The compounds of formulae (I), (IA) and (IB) may be investigated for inhibition of pro-inflammatory cytokines Interleukin-2, 4 and 13 in human CD4-positive T cells isolated from fresh isolated PBMCS using a CD4-positive T cell isolation kit.

CD4-positive T cells stimulated with beads coated with antibodies against CD2, CD3 and CD28 are incubated for 48 hours containing the compounds under investigation, reconstituted in DMSO. The secreted levels of Interleukin-2, 4 and 13 are measured in the cell culture supernatant using a cytometric bead array and the cell viability is measured using an MTT (3-(4,5-dimethylthiazol-2-yl)-2,5-diphenyltetrazolium bromide) assay.

Secreted IL-4 levels may be measured using electrochemiluminescence (MSD kits, Meso Scale Discovery), while secreted IL-2 levels may be measured using proximity homogenous time-resolved fluorescence (HTRF) and the amount of living cells may be measured by addition of resazurin (PrestoBlue®).

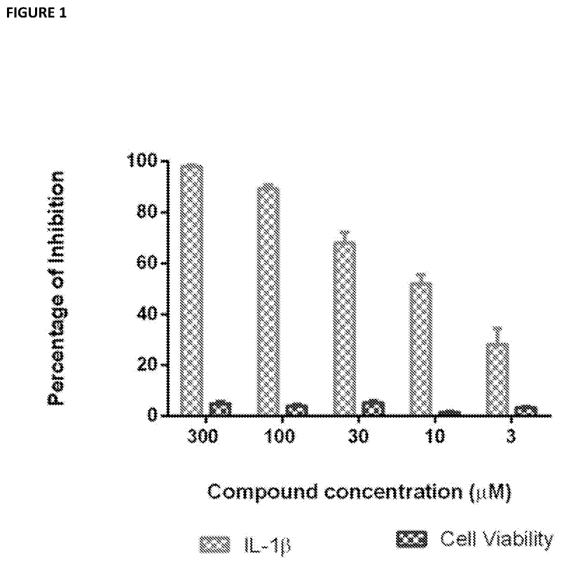

The compound 3,5-Diamino-6-(diphenylmethyl)-1,2,4-triazine, was investigated using the methods described above and was found to exhibit high inhibition of Interleukins IL-17A and IL-8, and a moderate inhibition of IL-1(3 and IL-6, as shown in FIGS. 1 to 6.

### 3,5-Diamino-6-(diphenylmethyl)-1,2,4-triazine

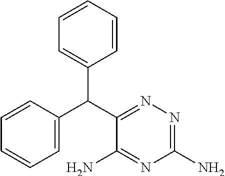 [SMILES: Nc1nnc(C(c2ccccc2)c2ccccc2)c(N)n1]

3,5-Diamino-6-(diphenylmethyl)-1,2,4-triazine may be prepared by the processes disclosed in WO2009/090431A1.

When tested in IL-2 and IL-4 inhibition assays, 3,5-Diamino-6-(diphenylmethyl)-1,2,4-triazine was found to exhibit relative EC50 of 28.0 and 50.5 nM respectively:

| | Rel EC50 | | Abs EC50 | | Max Effect |
|---|---|---|---|---|---|
| Inhibition of IL-2 | 28.0 | nM | 15.0 | nM | 97% |
| release | | | | | |
| Inhibition of IL-4 | 58.4 | nM | 61.3 | nM | 99% |
| release | | | | | |
| Inhibition of cell | >10000.0 | nM | >10000.0 | nM | 28% |
| viability | | | | | |

These data evidences a good level of inhibition of both IL-2 and IL-4 by 3,5-Diamino-6-(diphenylmethyl)-1,2,4-triazine without significant inhibition of cell viability.

Further data are shown in the Figures:

FIG. 1 illustrates the inhibitory effect of 3,5-diamino-6-[2-(3,4,5-trichlorothienyl)]-1,2,4-triazineon Interleukin (IL)-1 beta, wherein the bars indicate a mean±SEM for n=9-10 subjects.

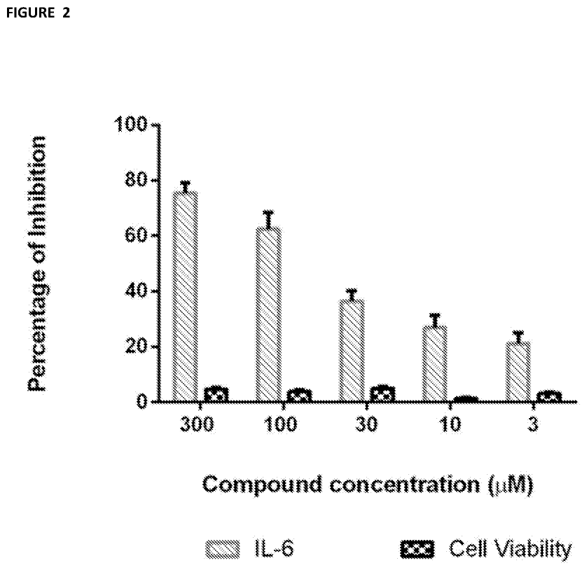

FIG. 2 illustrates the inhibitory effect of 3,5-diamino-6-[2-(3,4,5-trichlorothienyl)]-1,2,4-triazineon interleukin (IL)-6, wherein the bars indicate a mean±SEM for n=9-10 subjects.

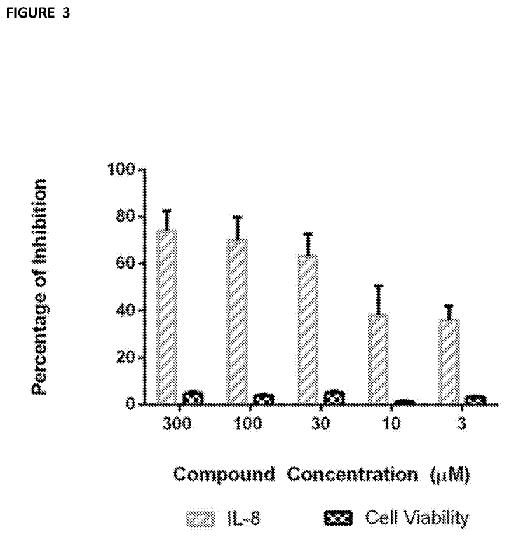

FIG. 3 illustrates the inhibitory effect of 3,5-diamino-6-[2-(3,4,5-trichlorothienyl)]-1,2,4-triazineon interleukin (IL)-8, wherein the bars indicate a mean±SEM for n=8-12 subjects.

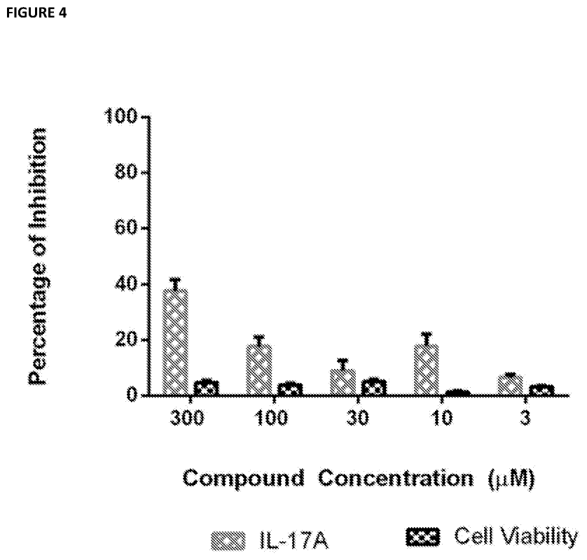

FIG. 4 illustrates the inhibitory effect of 3,5-diamino-6-[2-(3,4,5-trichlorothienyl)]-1,2,4-triazineon interleukin (IL)-17A, wherein the bars indicate a mean±SEM for n=5-7 subjects.

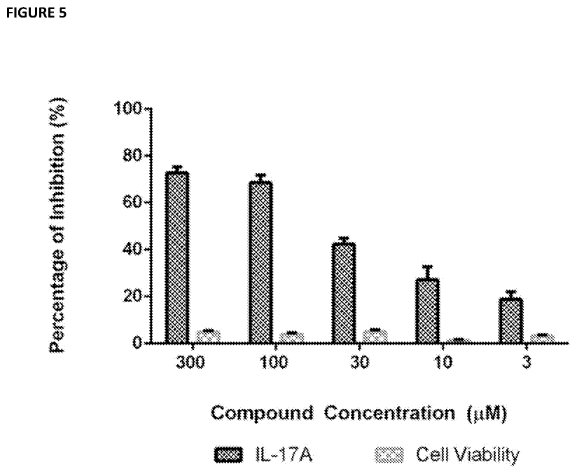

FIG. 5 illustrates the inhibitory effect of 3,5-Diamino-6-(diphenylmethyl)-1,2,4-triazine on interleukin (IL)-17A, wherein the bars indicate a mean±SEM for n=9-12 subjects.

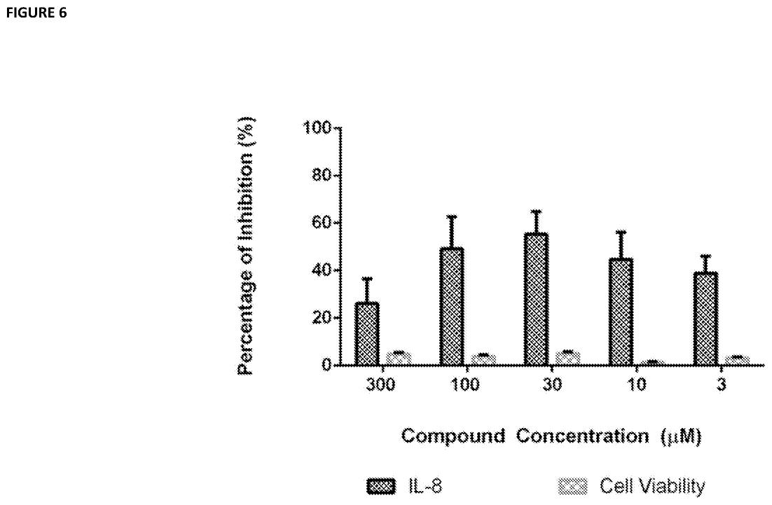

FIG. 6 illustrates the inhibitory effect of 3,5-Diamino-6-(diphenylmethyl)-1,2,4-on interleukin (IL)-8, wherein the bars indicate a mean±SEM for n=5-8 subjects.

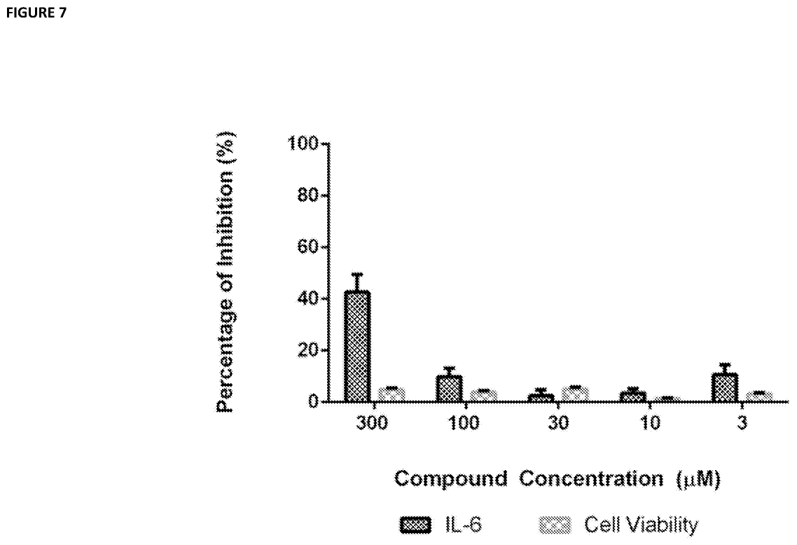

FIG. 7 illustrates the inhibitory effect of 3,5-Diamino-6-(diphenylmethyl)-1,2,4-on interleukin (IL)-6, wherein the bars indicate a mean±SEM for n=9-10 subjects.

1. A compound of the formula (I), or a salt, tautomer or solvate thereof; 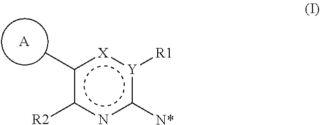
in which:
X is N and Y is C; or
X is C and Y is N; or
X and Y are both N:
A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆ alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups; or
A is a group 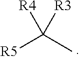
(wherein • indicates the point of attachment)
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;
R2 is amino, C₁₋₁₀ alkyl or phenyl;
R3 is phenyl, xanthyl or naphthyl, each optionally substituted with 1 to 5, preferably 2 to 5 substituents selected from halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups;
R5 is hydrogen;
and
N* is ═NH when R1 is hydrogen or a substituent group; or
N* is a group NRaRb where Ra and Rb are independently H or an alkyl group; or
N* is a piperazinyl ring, optionally substituted with one or more halogen or C₁-C₆alkoxy groups;
for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

2. A compound of Formula (I), for use as defined in claim 1, which is selected from:
3,5-Diamino-6-(2-thienyl)-1,2,4-triazine;
3,5-Diamino-6-(3-thienyl)-1,2,4-triazine;
3,5-Diamino-6-[3-(2,5 dichlorothienyl)]-1,2,4-triazine;
3,5-Diamino-6-[2-(3,4,5 trichlorothienyl)]-1,2,4-triazine;
5(3)-Amino-6-(2-thienyl)-2,3(2,5)-dihydro-3(5)-imino-2-methyl-1,2,4-triazine;
5(3)-Amino-6-(2-thienyl)-2,3(2,5)-dihydro-3(5)-imino-2-ethyl-1,2,4-triazine;
5(3)-Amino-6-[3-(2,5-dichlorothienyl)]-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-ethyl-1,2,4-triazine;
3,5-Diamino-6-[2-(4,5-dibromothienyl)]-1,2,4-triazine;
3,5-Diamino-6-[2-(5-bromothienyl)]-1,2,4-triazine;
3,5-Diamino-6-[2-(3-bromothienyl)]-1,2,4-triazine;
3,5-Diamino-6-[2-(5-chlorothienyl)]-1,2,4-triazine;
3,5-Diamino-6-[2-(benzo[b]thienyl)]-1,2,4-triazine;
3,5-Diamino-6-[2-(3-chlorobenzo[b]thienyl)]-1,2,4-triazine.
2,6-Diamino-3-(2-thienyl)-pyrazine;
2,4-Diamino-5-(2-thienyl)-pyrimidine;
2,6-Diamino-3-(3-thienyl)-pyrazine;
2,4-Diamino-5-(3-thienyl)-pyrimidine;
2,6-Diamino-3-[3-(2,5 dichlorothienyl)]-pyrazine;
2,4-Diamino-5-[3-(2,5 dichlorothienyl)]-pyrimidine;
2,6-Diamino-3-[2-(3,4,5 trichlorothienyl)]-pyrazine;
2,4-Diamino-5-[2-(3,4,5 trichlorothienyl)]-pyrimidine;
2(6)-Amino-3-(2-thienyl)-2,3(2,5)-dihydro-6(2)-imino-5-methyl-pyrazine;
4(2)-Amino-5-(2-thienyl)-2,3(2,5)-dihydro-2(4)-imino-1-methyl-pyrimidine;
2(6)-Amino-3-(2-thienyl)-2,3(2,5)-dihydro-6(2)-imino-5-ethyl-pyrazine;
4(2)-Amino-5-(2-thienyl)-2,3(2,5)-dihydro-2(4)-imino-1-ethyl-pyrimidine;
2(6)-Amino-3-[3-(2,5-dichlorothienyl)]-2,3(2,5)-dihydro-6(2)-imino-5-methyl-pyrazine;
4(2)-Amino-5-[3-(2,5-dichlorothienyl)]-2,3(2,5)-dihydro-2(4)-imino-1-methyl-pyrimidine;
2(6)-Amino-3-{2-(3,4,5-trichloro)thienyl}-2,3(2,5)-dihydro-6(2)-imino-5-methyl-pyrazine;
4(2)-Amino-5-{2-(3,4,5-trichloro)thienyl}-2,3(2,5)-dihydro-2(4)-imino-1-methyl-pyrimidine;
2(6)-Amino-3-{2-(3,4,5-trichloro)thienyl}-2,3(2,5)-dihydro-6(2)-imino-5-ethyl-pyrazine;
4(2)-Amino-5-{2-(3,4,5-trichloro)thienyl}-2,3(2,5)-dihydro-2(4)-imino-1-ethyl-pyrimidine;
2,6-Diamino-3-[2-(4,5-dibromothienyl)]-pyrazine;
2,4-Diamino-5-[2-(4,5-dibromothienyl)]-pyrimidine;
2,6-Diamino-3-[2-(5-bromothienyl)]-pyrazine;
2,4-Diamino-5-[2-(5-bromothienyl)]-pyrimidine;
2,6-Diamino-3-[2-(3-bromothienyl)]-pyrazine;
2,4-Diamino-5-[2-(3-bromothienyl)]-pyrimidine;
2,6-Diamino-3-[2-(5-chlorothienyl)]-pyrazine;
2,4-Diamino-5-[2-(5-chlorothienyl)]-pyrimidine;
2,6-Diamino-3-[2-(benzo[b]thienyl)]-pyrazine;
2,4-Diamino-5-[2-(benzo[b]thienyl)]-pyrimidine;
2,6-Diamino-3-[2-(3-chlorobenzo[b]thienyl)]-pyrazine;
2,4-Diamino-5-[2-(3-chlorobenzo[b]thienyl)]-pyrimidine;
3,5-Diamino-6-(diphenylmethyl)-1,2,4-triazine;
2,6-diamino-3-(diphenylmethyl)-pyrazine;
2,4-diamino-5-(diphenylmethyl)-pyrimidine;
3,5-Diamino-6-(1-cyclopentyl-1-phenyl-methyl)-1,2,4-triazine;
2,6-Diamino-3-(1-cyclopentyl-1-phenyl-methyl)-pyrazine;
2,4-Diamino-5-(1-cyclopentyl-1-phenyl-methyl)-pyrimidine;
3,5-Diamino-6-[1-(6-methoxynaphthalene)methyl]-1,2,4-triazine;
2,6-Diamino-3-[1-(6-methoxynaphthalene)methyl]-pyrazine;
2,4-Diamino-5-[1-(6-methoxynaphthalene)methyl]-pyrimidine;
3,5-Diamino-6-[1-(6-methoxynaphthalene)ethyl]-1,2,4-triazine;
2,6-Diamino-3-[1-(6-methoxynaphthalene)ethyl]-pyrazine;
2,4-Diamino-5-[1-(6-methoxynaphthalene)ethyl]-pyrimidine;
3,5-Diamino-6-(1-isopropyl-1-phenylmethyl)-1,2,4-triazine;
2,6-Diamino-3-(1-isopropyl-1-phenylmethyl)-pyrazine;
2,4-Diamino-5-(1-isopropyl-1-phenylmethyl)-pyrimidine;
3,5-Diamino-6-(9-xanthyl)-1,2,4-triazine;
2,6-Diamino-3-(9-xanthyl)-pyrazine;
2,4-Diamino-5-(9-xanthyl)-pyrimidine;
3,5-Diamino-6-[1,1 bis-(4-chlorophenyl)methyl]-1,2,4-triazine;
2,6-Diamino-3-[1,1 bis-(4-chlorophenyl)methyl]-pyrazine;
2,4-Diamino-5-[1,1 bis-(4-chlorophenyl)methyl]-pyrimidine;
3,5-Diamino-6-[1,1-bis-(4-fluorophenyl)methyl]-1,2,4-triazine;
2,6-Diamino-3-[1,1-bis-(4-fluorophenyl)methyl]-pyrazine;
2,4-Diamino-5-[1,1-bis-(4-fluorophenyl)methyl]-pyrimidine;
3,5-Diamino-6-{1-(4-chlorophenoxy)-1-methyl}ethyl-1,2,4-triazine;
2,6-Diamino-3-{1-(4-chlorophenoxy)-1-methyl}ethyl-pyrazine; and
2,4-Diamino-5-{1-(4-chlorophenoxy)-1-methyl}ethyl-pyrimidine;
or a salt, tautomer or solvate thereof.

3. A compound of the formula (IA), or a salt, tautomer or solvate thereof: 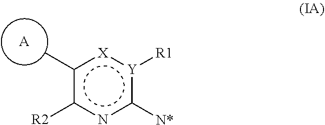
in which:
X is N and Y is C; or
X is C and Y is N
A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆ alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups;
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;
R2 is amino, C₁₋₁₀ alkyl or phenyl;
and
N* is ═NH when R1 is hydrogen or a substituent group; or
N* is a group NRaRb where Ra and Rb are independently H or an alkyl group; or
N* is a piperazinyl ring, optionally substituted with one or more halogen or C₁-C₆alkoxy groups;
provided that when X is C and Y is N, and A is thienyl optionally substituted with halogen, then R2 is not C₁-C₃ alkyl.

4. A compound of Formula (IA), which is selected from:
2,6-Diamino-3-(2-thienyl)-pyrazine;
2,4-Diamino-5-(2-thienyl)-pyrimidine;
2,6-Diamino-3-(3-thienyl)-pyrazine;
2,4-Diamino-5-(3-thienyl)-pyrimidine;
2,6-Diamino-3-[3-(2,5 dichlorothienyl)]-pyrazine;
2,4-Diamino-5-[3-(2,5 dichlorothienyl)]-pyrimidine;
2,6-Diamino-3-[2-(3,4,5 trichlorothienyl)]-pyrazine;
2,4-Diamino-5-[2-(3,4,5 trichlorothienyl)]-pyrimidine;
2(6)-Amino-3-(2-thienyl)-2,3(2,5)-dihydro-6(2)-imino-5-methyl-pyrazine;
4(2)-Amino-5-(2-thienyl)-2,3(2,5)-dihydro-2(4)-imino-1-methyl-pyrimidine;
2(6)-Amino-3-(2-thienyl)-2,3(2,5)-dihydro-6(2)-imino-5-ethyl-pyrazine;
4(2)-Amino-5-(2-thienyl)-2,3(2,5)-dihydro-2(4)-imino-1-ethyl-pyrimidine;
2(6)-Amino-3-[3-(2,5-dichlorothienyl)]-2,3(2,5)-dihydro-6(2)-imino-5-methyl-pyrazine;
4(2)-Amino-5-[3-(2,5-dichlorothienyl)]-2,3(2,5)-dihydro-2(4)-imino-2-methyl-pyrimidine;
2(6)-Amino-3-{2-(3,4,5-trichloro)thienyl}-2,3(2,5)-dihydro-6(2)-imino-5-methyl-pyrazine;
4(2)-Amino-5-{2-(3,4,5-trichloro)thienyl}-2,3(2,5)-dihydro-2(4)-imino-1-methyl-pyrimidine;
2(6)-Amino-3-{2-(3,4,5-trichloro)thienyl}-2,3(2,5)-dihydro-6(2)-imino-5-ethyl-pyrazine;
4(2)-Amino-5-{2-(3,4,5-trichloro)thienyl}-2,3(2,5)-dihydro-2(4)-imino-2-ethyl-pyrimidine;
2,6-Diamino-3-[2-(4,5-dibromothienyl)]-pyrazine;
2,4-Diamino-5-[2-(4,5-dibromothienyl)]-pyrimidine;
2,6-Diamino-3-[2-(5-bromothienyl)]-pyrazine;
2,4-Diamino-5-[2-(5-bromothienyl)]-pyrimidine;
2,6-Diamino-3-[2-(3-bromothienyl)]-pyrazine;
2,4-Diamino-5-[2-(3-bromothienyl)]-pyrimidine;
2,6-Diamino-3-[2-(5-chlorothienyl)]-pyrazine;
2,4-Diamino-5-[2-(5-chlorothienyl)]-pyrimidine;
2,6-Diamino-3-[2-(benzo[b]thienyl)]-pyrazine;
2,4-Diamino-5-[2-(benzo[b]thienyl)]-pyrimidine;
2,6-Diamino-3-[2-(3-chlorobenzo[b]thienyl)]-pyrazine; and
2,4-Diamino-5-[2-(3-chlorobenzo[b]thienyl)]-pyrimidine;
or a salt, tautomer or solvate thereof.

5. A pharmaceutical composition comprising a compound of Formula (IA), or a salt, tautomer or solvate thereof, as defined in claim 3 or claim 4, and a pharmaceutically acceptably excipient.

6. A compound of Formula (IA), or a salt, tautomer or solvate thereof, as defined in claim 3 or 4, for use as a medicament.

7. A compound of Formula (IA), or a salt, tautomer or solvate thereof, as defined in claim 3 or claim 4, for use in the treatment of a disorder or condition selected from for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis; or epilepsy; multiple sclerosis; glaucoma and uveitis; cerebral traumas and cerebral ischaemias; stroke, head injury; spinal cord injury; surgical trauma; neurodegenerative disorders; motor neurone disease; Alzheimer's disease; Parkinson's disease; chronic inflammatory pain; neuropathic pain; migraine; bipolar disorder; mood, anxiety and cognitive disorders; schizophrenia; and trigeminal autonomic cephalalgias.

8. A compound of Formula (IB), or a salt, tautomer or solvate thereof: 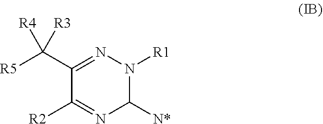
in which
R3 is phenyl, xanthyl or naphthyl, each optionally substituted with 1 to 5 substituents selected from halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 1 to 5 substituents selected from halogen or C₁-C₆alkoxy groups;
R5 is hydrogen;
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;
R2 is amino, C₁₋₁₀ alkyl or phenyl;
N* is amino when R1 is hydrogen or ═NH when R1 is a substituent group; or
N* is a group NRaRb where Ra and Rb are independently H or an alkyl group; or
N* is a piperazinyl ring, optionally substituted with one or more halogen or C₁-C₆alkoxy groups;
for use in the treatment of a disorder or condition selected from asthma, solid organ transplantation rejection, atopic dermatitis, eczema, Hodgkins Disease, psoriasis, ankylosing spondylitis, rheumatoid arthritis, psoriatic arthritis, Metastatic melanoma, Renal Cell carcinoma, Colorectal cancer, non-Hodgkin's lymphoma, Melanoma, Metastatic Renal Cancer, Breast cancer, Colon cancer, Renal cell cancer, Cancer metastatic growth in lung and liver, chronic obstructive pulmonary disease (COPD), and Pulmonary tuberculosis.

9. A compound of Formula (IB) for use as defined in claim 8, wherein R3 is phenyl, xanthyl or naphthyl, each optionally substituted with 2 to 5 substituents selected from halogen or C₁-C₆alkoxy groups.

10. A compound of formula (IB), for use as defined in claim 8, which is selected from
3,5-Diamino-6-(diphenylmethyl)-1,2,4-triazine;
3,5-Diamino-6-(1-cyclopentyl-1-phenyl-methyl)-1,2,4-triazine;
3,5-Diamino-6-[1-(6-methoxynaphthalene)methyl]-1,2,4-triazine;
3,5-Diamino-6-[1-(6-methoxynaphthalene)ethyl]-1,2,4-triazine;
3,5-Diamino-6-(1-isopropyl-1-phenylmethyl)-1,2,4-triazine
3,5-Diamino-6-(9-xanthyl)-1,2,4-triazine;
3,5-Diamino-6-[1,1 bis-(4-chlorophenyl)methyl]-1,2,4-triazine;
3,5-Diamino-6-[1,1-bis-(4-fluorophenyl)methyl]-1,2,4-triazine;
and
3,5-Diamino-6-{1-(4-chlorophenoxy)-1-methyl}ethyl-1,2,4-triazine; or
a salt, tautomer or solvate thereof.
